(19) United States
(12) Patent Application Publication
(10) Pub. No.: US 2011/0125057 A1
(43) Pub. Date: May 26, 2011

(54) INTRAVENOUS CANNULA ASSEMBLY

(76) Inventors: Allan Walsh, Medowie (AU); Feiyan Tan, Guangdong (CN)

(21) Appl. No.: 12/447,656

(22) PCT Filed: Sep. 28, 2007
(86) PCT No.: PCT/AU07/01438

§ 371 Date: Jan. 21, 2011

(30) Foreign Application Priority Data
Sep. 28, 2006 (AU) 2006905370

(51) Int. Cl. A61B 5/15

(52) U.S. Cl. 600/573

(57) ABSTRACT

An intravenous cannula assembly comprises a longitudinally extending barrel (10, 210), a cannula hub (30, 230), a cannula (50, 250), a cannula guard (70, 270) and a deployment mechanism (90, 100, 290, 300). The cannula hub (30, 230) is mounted in the barrel (10, 210) and has a hub aperture (35, 235) extending between the hub leading end (31, 231) and hub trailing end (32, 232). The cannula hub (30, 230) is adapted to mount a tube at the hub trailing end (32, 232) in fluid communication with the hub aperture (35, 235). The cannula (50, 250) is mounted in, and in fluid communication with, the hub aperture (35, 235). The cannula (50, 250) extends from the hub leading end (31, 231) through the barrel leading end (11, 211). The cannula guard (70, 270) is concentrically mounted about the cannula hub (30, 230). The deployment mechanism (90, 100, 290, 300) is adapted to deploy the cannula guard (70, 270) from a retracted position substantially within the barrel (10, 210) to a deployed position extending from the barrel leading end (11, 211) and covering the portion of the cannula (50, 250) extending through the barrel leading end (11, 211).

### TECHNICAL FIELD

The present invention relates to the field of intravenous cannula assemblies and in particular relates to, but is not limited to, a blood bag needle assembly.

### BACKGROUND OF THE INVENTION

Typical blood bag systems for use in extracting blood from blood donors consist of a blood bag for storing the extracted blood, a flexible delivery tube fixed at one end to (and communicating with) the blood bag and a needle fixed to (and communicating with) the opposing end of the delivery tube. A needle cap is mounted on the needle and a needle cover is typically loosely mounted on the tube. In use, the medical practitioner firstly removes the needle cap so as to expose the needle. The needle is inserted into the donor's arm and blood extracted from the donor into the blood bag via the delivery tube. Once the blood extraction is complete, the delivery tube is clamped and the needle is removed from the donor's arm. The removed needle bears the donor's blood and freely dangles at the end of the flexible tube. Whilst the cover located on the tube is intended to be slipped down over the needle for protection, this is a relative clumsy procedure. Prior to successfully covering the needle, there is a risk of spreading blood-borne diseases. The medical practitioner or other personnel involved in processing the collected blood may inadvertently prick themselves with the needle or wipe blood on the exterior of the needle onto their skin. If the donor's blood is infected, the medical practitioner/other personnel may become infected. Conversely, if the medical practitioner/other personnel is carrying a blood-borne disease, the collected blood may become infected. Similar risks exist in other types of intravenous cannula assembly applications.

### OBJECT OF THE INVENTION

It is the object of the present invention to substantially overcome or at least ameliorate one or more of the above disadvantages.

### SUMMARY OF THE INVENTION

There is disclosed herein an intravenous cannula assembly comprising:

a longitudinally extending barrel having a barrel leading end and a barrel trailing end;

a cannula hub mounted in said barrel and having a hub leading end, a hub trailing end and a hub aperture extending between said hub leading end and said hub trailing end, said cannula hub being adapted to mount a tube at said hub trailing end in fluid communication with said hub aperture;

a cannula mounted in, and in fluid communication with, said hub aperture, said cannula extending from said hub leading end through said barrel leading end;

a cannula guard concentrically mounted about said cannula hub; and

a deployment mechanism adapted to deploy said cannula guard from a retracted position at least substantially within said barrel to a deployed position extending from said barrel leading end and covering the portion of said cannula extending through said barrel leading end.

Typically, said deployment mechanism comprises:

a spring biasing said cannula guard towards said deployed position; and

a releasable locking arrangement adapted to retain said cannula guard in said retracted position until release of said releasable locking arrangement.

In a preferred form, said releasable locking arrangement comprises:

a first interlocking structure provided on said cannula guard;

a locking member mounted on said barrel and having a second interlocking structure;

said locking member being displaceable between a locking position at which said first interlocking structure engages said second interlocking structure to thereby retain said cannula guard in said retracted position and an unlocking position at which said first interlocking structure disengages from said second interlocking structure, thereby releasing said cannula guard.

Typically, said locking member is of a generally cylindrical form and is angularly displaceable about a longitudinal axis of said barrel between said locking position and said unlocking position.

The locking member is generally mounted on said barrel adjacent said barrel trailing end.

In one embodiment, said barrel has a pair of opposing substantially flat faces, said locking member protruding laterally beyond each of said faces.

Preferably, said cannula hub is mounted on said locking member.

Typically, said cannula hub and said cannula are each longitudinally fixed in relation to said barrel.

Typically, said cannula guard is rotationally fixed in relation to said barrel.

Preferably, said assembly further comprises a captive locking arrangement adapted to captively retain said cannula guard in said deployed position after deployment.

Typically, said captive locking arrangement comprises co-operating structures of said cannula guard and of said barrel configured to allow relative displacement of said cannula guard to said deployed position and to prevent relative displacement of said cannula guard from said deployed position towards said retracted position.

Typically, said captive locking arrangement comprises:

a pair of tabs provided on opposing sides of said cannula guard, each said tab having a leading edge hingedly connected to a stem of said cannula guard and biased to protrude from said stem; and

a co-operating structure of said barrel adapted to engage said tabs, deflecting said tabs inwardly, as said cannula guard is deployed toward said deployed position, and to engage a trailing end of each said tab when said cannula guard is in said deployed position, preventing displacement of said cannula guard from said deployed position toward said retracted position.

In one embodiment, said co-operating structure of said barrel comprises a neck of said barrel defining a reduced cross-section leading portion of said barrel aperture.

In an alternative embodiment, said co-operating structure of said barrel comprises a pair of opposing detents projecting into said barrel aperture.

In one form, said captive locking arrangement further comprises an external shoulder formed on said cannula guard toward a trailing end of said cannula guard, said shoulder being adapted to engage a co-operating structure of said barrel to prevent said cannula guard from being deployed beyond said deployed position.

### BRIEF DESCRIPTION OF THE DRAWINGS

Preferred embodiments of the present invention will now be described, by way of an example only, with reference to the accompanying drawings wherein:

FIG. 1 is a partially cross-sectioned front elevation view of an intravenous cannula assembly according to a first embodiment with the cannula guard in a retracted position;

FIG. 2 is a partially cross-sectioned front elevation view of the cannula assembly of FIG. 1 with the cannula guard in a deployed position;

FIG. 3 is a front elevation view of the cannula hub of the cannula assembly of FIG. 1;

FIG. 4 is a cross-sectional view of the cannula hub of FIG. 3 taken through section 4-4;

FIG. 5 is a front elevation view of the cannula guard of the cannula assembly of FIG. 1;

FIG. 6 is a left side elevation view of the cannula guard of FIG. 5;

FIG. 7 is a cross-sectional view of the cannula guard of FIG. 5 taken through section 7-7;

FIG. 8 is a leading end view of the cannula guard of FIG. 5;

FIG. 9 is a trailing end view of the cannula guard of FIG. 5;

FIG. 10 is a front elevation view of the barrel of the cannula assembly of FIG. 1;

FIG. 11 is a side elevation view of the barrel of FIG. 10;

FIG. 12 is a leading end view of the barrel of FIG. 10;

FIG. 13 is a trailing end view of the barrel of FIG. 10;

FIG. 14 is a cross-sectional front elevation view of the barrel of FIG. 10 taken through section 14-14;

FIG. 15 is a cross-sectional side elevation view of the barrel of FIG. 10 taken through section 15-15;

FIG. 16 is a front elevation of the locking member of the cannula assembly of FIG. 1;

FIG. 17 is a leading end view of the locking member of FIG. 16;

FIG. 18 is a trailing end view of the locking member of FIG. 16;

FIG. 19 is a front elevation cross-sectional view of the locking member of FIG. 16 taken through section 19-19;

FIG. 20 is a cross-sectional view of the locking member of FIG. 16 taken through section 20-20;

FIG. 21 is a schematic partially cross-sectioned view of the interlocking structures of the cannula guard of FIG. 5 and locking member of FIG. 16;

FIG. 22 is a front elevation view of a cannula cap for use with the cannula assembly of FIG. 1;

FIG. 23 is a cross-sectional view of the cannula cap of FIG. 22 taken through section 22-22.

FIG. 24 is a partially cross-sectioned front elevation view of an intravenous cannula assembly according to a second embodiment with the cannula guard in a retracted position;

FIG. 25 is a partially cross-sectioned front elevation view of the cannula assembly of FIG. 24 with the cannula guard in a deployed position;

FIG. 26 is an inverse plan view of the cannula assembly of FIG. 24;

FIG. 27 is a front elevation view of the cannula hub of the cannula assembly of FIG. 24;

FIG. 28 is a plan view of the cannula hub of FIG. 27;

FIG. 29 is a cross-sectional front elevation view of the cannula guard of the cannula assembly of FIG. 24;

FIG. 30 is a side elevation view of the cannula guard of FIG. 29;

FIG. 31 is a perspective view of the cannula guard of FIG. 29;

FIG. 32 is a front elevation view of the barrel of the cannula assembly of FIG. 24;

FIG. 33 is a cross-sectional front elevation view of the barrel of FIG. 32;

FIG. 34 is a plan view of the barrel of FIG. 32;

FIG. 35 is an inverse plan view of the barrel of FIG. 32;

FIG. 36 is a plan view of the locking member of the cannula assembly of FIG. 24;

FIG. 37 is an inverse plan view of the locking member of FIG. 36;

FIG. 38 is a perspective view of the locking member of FIG. 36; and

FIG. 39 is a cross-sectional view of the locking member of FIG. 36.

### DETAILED DESCRIPTION OF THE PREFERRED EMBODIMENTS

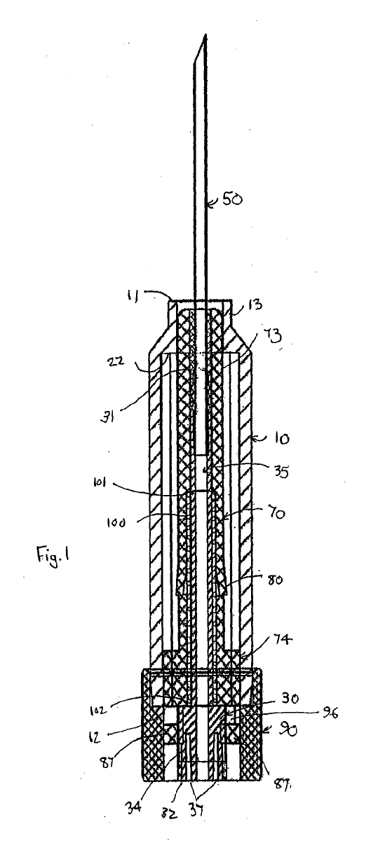

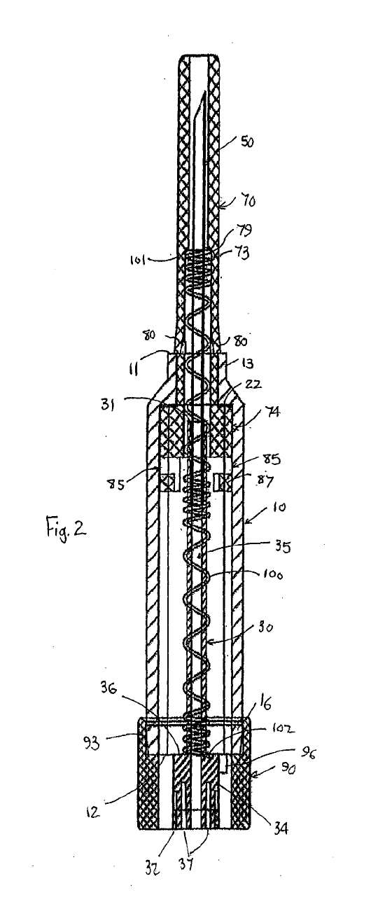

Referring firstly to FIGS. 1 and 2 of the accompanying drawings, an intravenous cannula assembly 1 according to a first embodiment, in the form of a blood bag needle assembly, generally comprises a barrel 10, cannula hub 30, cannula 50, cannula guard 70 and deployment mechanism which here includes a locking member 90 and spring 100. The barrel 10 has a barrel leading end 11 and a barrel trailing end 12. The cannula hub 30 has a hub leading end 31, a hub trailing end 32 and a hub aperture 35 extending therebetween. The cannula 50, here in the form of a blood bag needle, is mounted in, and in fluid communication with, the hub aperture 35 and extends from the hub leading end 31 through the barrel leading end 11. The cannula guard 70 is concentrically mounted about the cannula hub 30. The deployment mechanism is operable to deploy the cannula guard 70 from a retracted position, depicted in FIG. 1, at which it is at least substantially located within the barrel 10, to a deployed position, depicted in FIG. 2, extending from the barrel leading end 11 and covering the portion of the cannula 50 that extends through the barrel leading end 11. The barrel 10, cannula hub 30, cannula guard 70 and locking member 90 will typically be moulded from plastic material. The needle 50 and spring 100 will typically be formed from stainless steel.

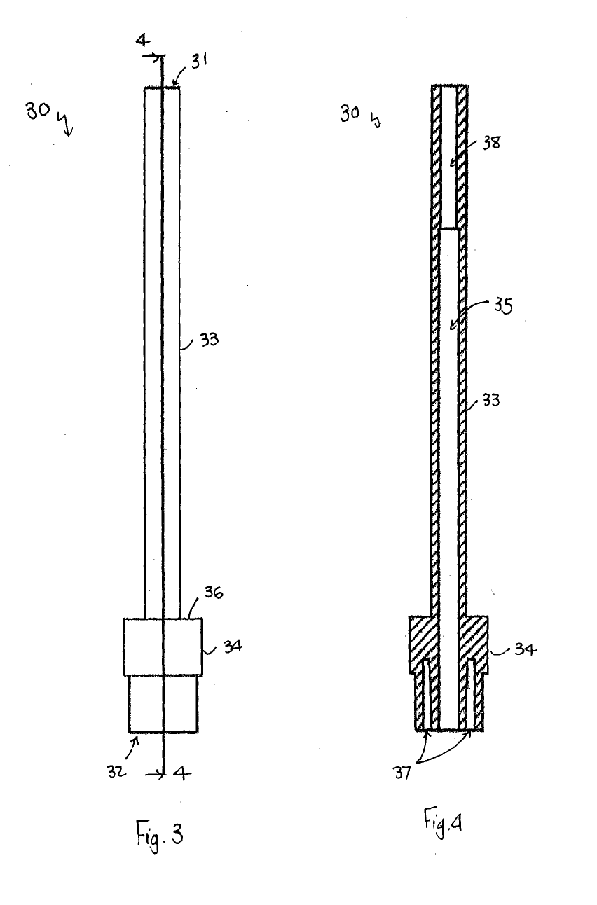

The cannula hub 30 of the first embodiment is depicted in detail in FIGS. 3 and 4. The cannula hub 30 comprises an elongate tubular hub stem 33 located towards the hub leading end 31 and a stepped hub boss 34 located toward the hub trailing end 32. The hub aperture 35 extends throughout the length of the cannula hub 30 from the hub leading end 31 to the hub trailing end 32. An annular shoulder 36 is defined on the leading face of the hub boss 34 where it meets the trailing end of the hub stem 33. An annular recess 37 is formed in the trailing face of the hub boss 34 for receipt of a blood delivery tube (not depicted) such that the blood delivery tube is in fluid communication with the hub aperture 35 (and thus the needle 50). The annular recess 37 is sized so as to provide an interference fit with the annular wall of the blood delivery tube such that it remains fixed to the cannula hub 30. Alternatively, the cannula hub 30 could be overmoulded on to the end of the blood delivery tube. The leading portion 38 of the hub aperture 35 is slightly narrower than the remainder of the hub aperture 35, and is sized to receive the needle 50, again with an interference fit so that the needle 50 remains fixed in relation to the cannula hub 30.

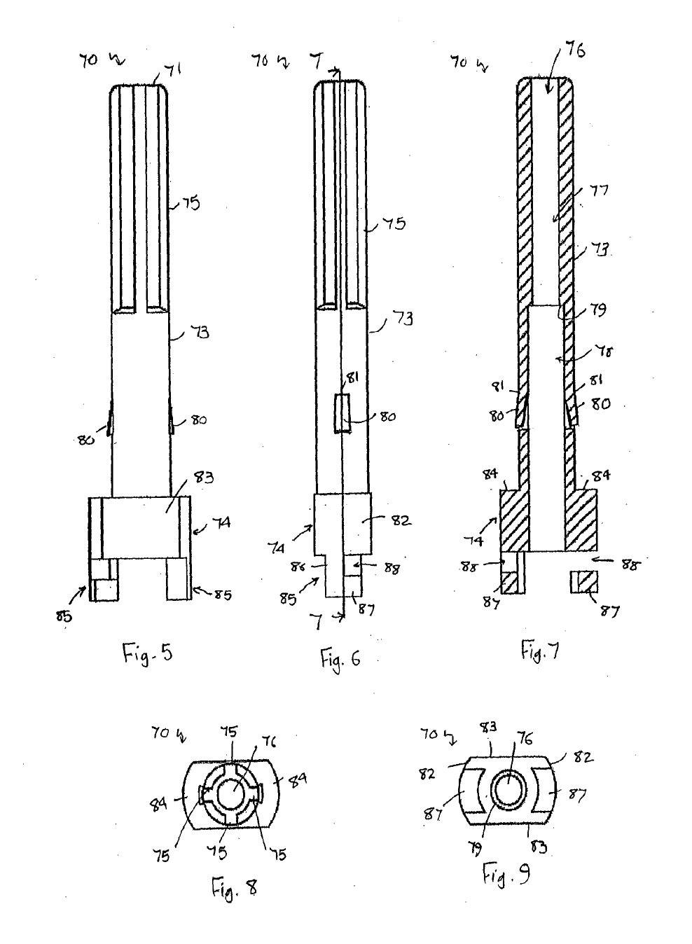

The cannula guard 70 of the first embodiment is depicted in detail in FIGS. 5 through 9. The cannula guard 70 has a leading end 71 and trailing end 72. The cannula guard 70 comprises an elongate tubular guard stem 73 located towards the guard leading end 71 and an enlarged guard head 74 located toward the guard trailing end 72. The guard stem 73 has a generally cylindrical configuration, however the leading portion is configured with a series of four elongate ribs 75 equally spaced about the circumference of the guard stem 73, providing the same effective diameter as the remainder of the guard stem 73. This configuration reduces weight whilst maintaining the same effective external diameter throughout the length of the guard stem 73. A guard aperture 76 extends though the length of the cannula guard 70 from the guard leading end 71 through to the guard trailing end 72. The guard aperture 76 has a leading portion 77, sized to receive the hub stem 33 and a trailing portion 78 of greater diameter sized to receive both the hub stem 33, and the spring 100 concentrically mounted on the hub stem 33, as depicted in FIGS. 1 and 2. An annular internal shoulder 79 is defined at the intersection between the leading portion 77 and trailing portion 78 of the guard aperture 76.

Two diametrically opposed tabs 80 are provided on the guard stem 73. The tabs 80 are cut out from the annular wall of the guard stem 73 on their lateral and trailing sides, remaining effectively hinged from the stem 73 at their leading edges 81. The tabs 80 are biased to protrude from the stem 73, as best depicted in FIG. 7.

Referring to FIG. 9, the guard head 74 has a peripheral cross-section defined by two opposing arcs joined by two opposing parallel lines, providing two opposing part cylindrical walls 82 and two opposing flattened side walls 83. Two external shoulders 84 are defined at the intersection between the guard stem 73 and the guard head 74, between the guard stem 73 and each opposing part cylindrical wall 82 of the leading face of each guard head 74. Two hook-like members 85 are formed on the trailing face of the guard head 74. Each of the hook-like members 85 comprises a longitudinally extending upright 86 extending from the guard head 74 and a laterally extending flange 87 extending at right angles to the upright 86. A locking recess 88 is defined between the leading face of each flange 87 and the trailing face of the guard head 74. When viewed from the trailing end, as in FIG. 9, each flange 87 generally extends in a clock wise direction about the longitudinal axis of the cannula guard 70 from the respective upright 86.

The body 10 of the first embodiment is depicted in detail in FIGS. 10 through 15. The body 10 is in the general form of a barrel and comprises a cylindrical neck 13 at the body leading end 11, a barrel 14 having a generally cylindrical configuration with two flattened opposing side walls 15 and a cylindrical tail 16 at the barrel trailing end 12. The tail 16 has the same diameter as the major diameter of the body 14. A tapered shoulder 17 connects the neck 13 and body 14. A barrel aperture 18 extends through the length of the barrel 10 from the barrel leading end 11 to the barrel trailing end 12. A leading portion 19 of the barrel aperture 18, defined by the neck, 13 has a reduced cross-section, being cylindrical, with a diameter sized to receive the guard stem 73. The trailing portion 20 of the barrel aperture 18, located within the body 14 and tail 16, has two opposing flattened side walls 21, as best depicted in FIG. 13. The cross section of the trailing portion 20 of the barrel aperture 18 matches that of the guard head 74 (best depicted in FIG. 9) so as to prevent rotation of the cannula guard 70 within the barrel 10. Two opposing internal shoulders 22 are defined between the leading and trailing portions 19, 20 of the barrel aperture 18.

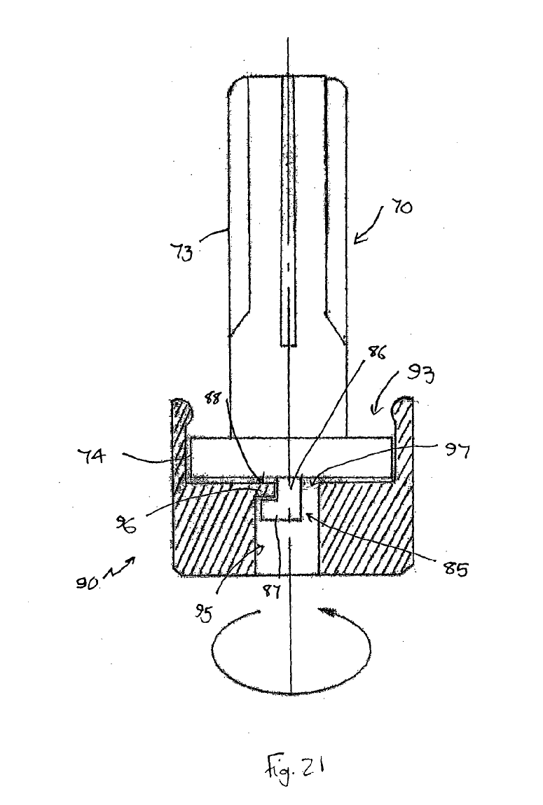

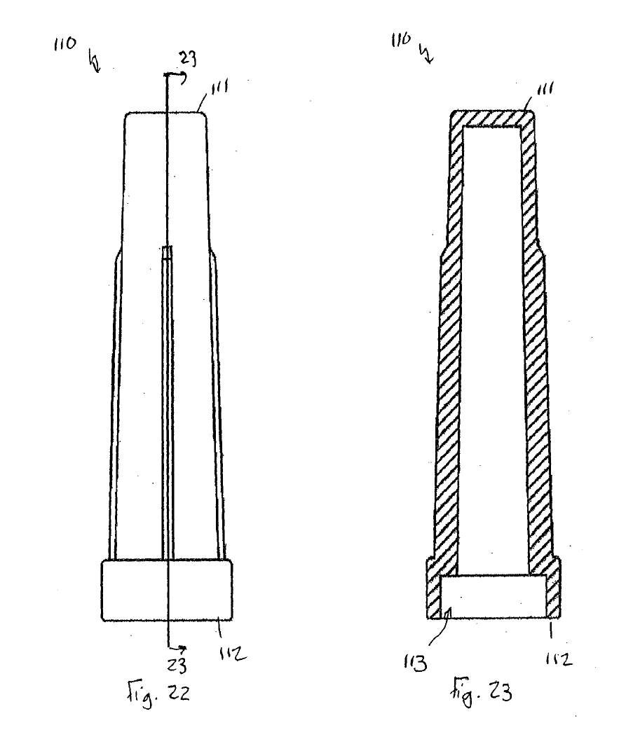

The deployment mechanism includes the spring 100, which biases the cannula guard 70 towards its deployed position depicted in FIG. 2, and a releasable locking arrangement which retains the cannula guard 70 in the retracted position, as depicted in FIG. 1, until release of the releasable locking arrangement. The releasable locking arrangement here comprises a first interlocking structure provided on the cannula guard 70, in the form of the hook-like members 85, and a locking member 90. The locking member 90 is depicted in detail in FIGS. 16 through 20.

The locking member 90 has a generally cylindrical form, including a knurled peripheral surface 98 to facilitate grip. The locking member 90 has a leading end 91 and trailing end 92. A large cylindrical recess 93 is formed at the leading end 91 of the locking member 90 for receipt of the tail 16 of the barrel 10. An inwardly projecting annular rim 98 is formed in the wall of the recess 93 to lock the tail 16 of the barrel 10 into the recess 93 whilst allowing relative rotation between the barrel 10 and locking member 90 with the locking member 90 being angularly displaceable about the longitudinal axis of the barrel 10. A central locking member aperture 94 extends through the thickness of the locking member 90 for receipt of the hub boss 34. There is an interference fit between the wall of the locking member aperture 94 and the wider portion of the hub boss 34 to fix the cannula hub 30 to the locking member 90.

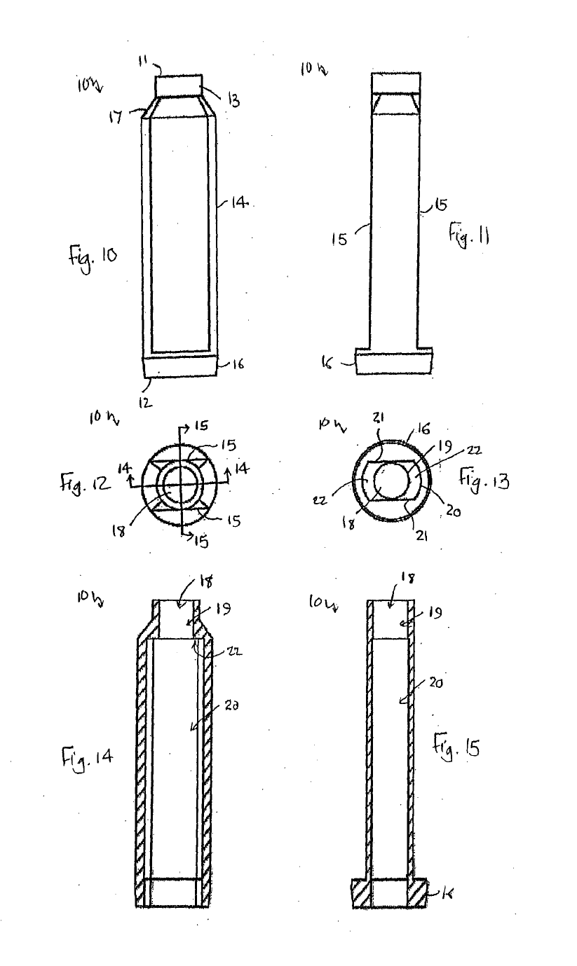

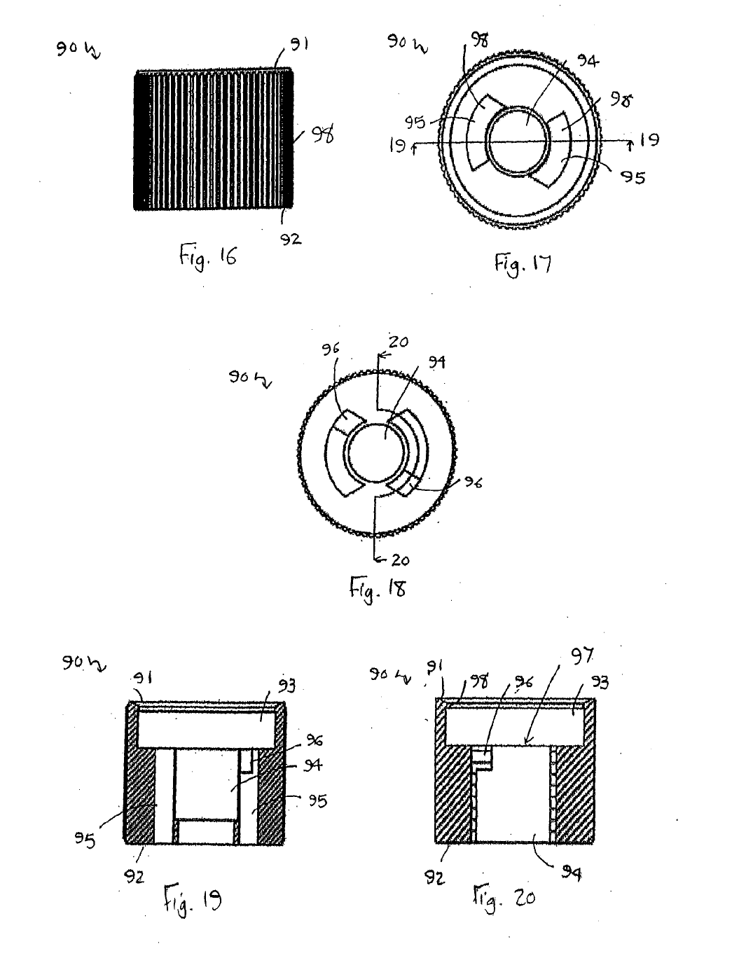

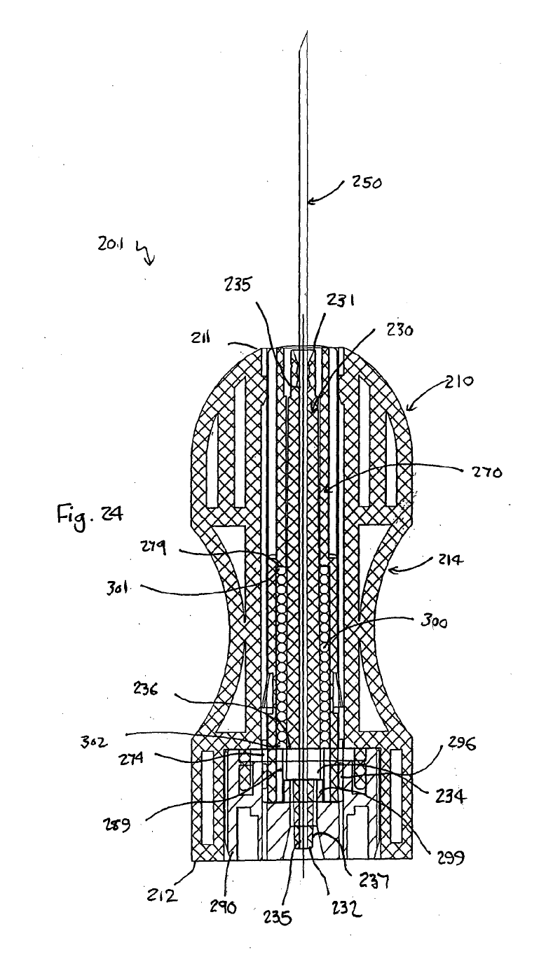

Two opposing curved locking cavities 95 extend from the trailing end 92 to the recess 93, on diametrically opposing sides of the central cap aperture 94 as best depicted in FIGS. 17 and 18. A tangentially extending locking projection 96 extends from one end of each of the locking cavities 95 adjacent the recess 93, as best depicted in FIGS. 18 and 20. The locking projections 96 form a second interlocking structure configured to engage the first interlocking structure defined by the hook-like members 85 of the cannula guard 70 as schematically depicted in FIG. 21. The shape and size of the opening 97 of the locking cavities 95 at the intersection with the recess 93 matches the shape of the flanges 87 of the hook-like members 85 on the guard head 74. The locking projections 96 are formed at opposing ends of the locking apertures 95, as best depicted in FIG. 18. The cross section of the locking projections 96, as depicted in FIGS. 20 and 21, matches the cross section of the recesses 88 defined by the hook-like members 85, as depicted in FIGS. 6 and 21. The configuration is such that, with the hook-like members 85 inserted into the locking cavities 95 by way of the openings 97, the locking projections 96 will be received in the locking recesses 88 when the locking member 90 is rotated counter-clockwise in a locking position when viewed from the trailing end. The resulting engagement between the leading face of the flanges 87 of the hook-like members 85 and the trailing face of the locking projections 96, will retain the cannula guard 70 in the retracted position. As an alternative to the interlocking configuration of the hook-like members 85 and locking projections 96, opposing inclined ramp surfaces that engage when the locking member 90 is in the locking position are envisaged.

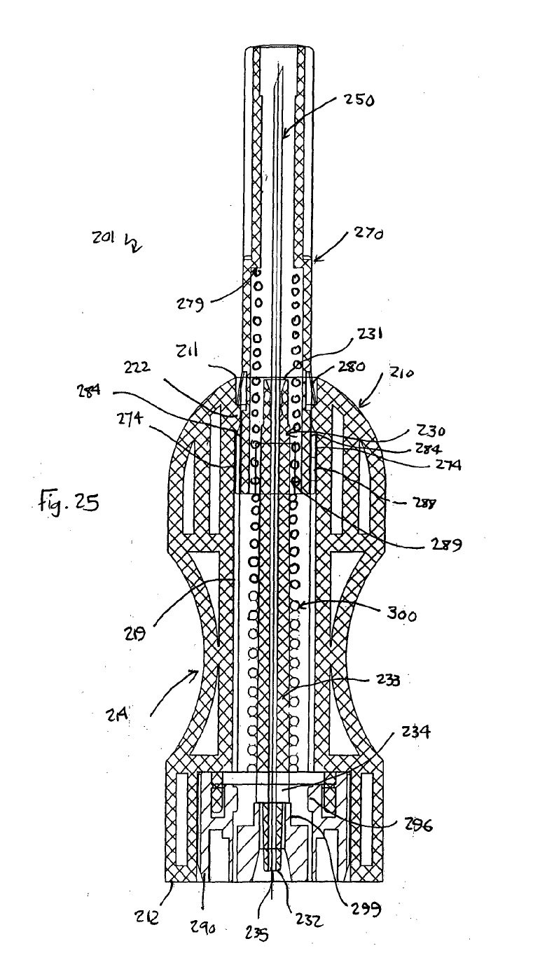

Referring to FIGS. 22 and 23, the cannula assembly 1 will also typically be provided with a standard needle cap 110 having a closed leading end 111 and open trailing end 112 designed to be fitted over the needle 50. The recess 113 in the open trailing end 112 is mounted on the neck portion 13 of the barrel 10 prior to use of the cannula assembly.

Referring back to FIG. 1, the cannula assembly 1 is assembled with the cannula guard 70 in the retracted position. The needle 50 is mounted in the hub aperture 35 of the cannula hub 30, extending from the hub leading end 31 through the barrel leading end 11. The cannula hub 30 is in turn mounted in the barrel 10 with the hub boss 34 located within the locking member aperture 94 of the locking member 90. The locking member 90 is mounted on the trailing end of the barrel 10, with the tail 16 of the barrel 10 locked into the recess 93 of the locking member 90. The blood delivery tube (not depicted) is fixed to the hub boss 34 by way of the annular recess 37 in the trailing face of the hub boss 34. The cannula guard 70 is concentrically mounted about the cannula hub 30 within the barrel 10 in the retracted position with the leading end 71 of the cannula guard 70 located within the neck 13 of the barrel 10. The hook-like members 85 of the cannula guard 70 extend into the locking cavities 95 of the locking member 90, with the locking tabs 96 received in the recesses 88 defined by the hook-like members 85, thereby retaining the cannula guard 70 in the retracted position. The spring 100 is concentrically mounted about the hub stem 33 of the cannula hub 30 within the guard aperture 76 of the cannula guard 70. The leading end 101 of the spring 100 bears against the shoulder 79 within the guard aperture 76, whilst the trailing end 102 of the spring 100, bears against the shoulder 36 defined on the leading face of the hub boss 34. The spring 100 imparts a driving force on the cannula guard 70 tending to deploy it towards the deployed position, however deployment is prevented by virtue of engagement of the hook-like members 85 of the cannula guard 70 and the locking projections 96 of the locking member 90. The needle cap 110 is mounted over the needle 50 with the recess 113 located on the neck 13 of the barrel 10.

In use, the cannula assembly will be communicated with a blood bag utilising a flexible blood delivery tube, connected to the cannula assembly 1 by way of the annular recess 37 in the rear face of the hub boss 34. The needle cap 110 is then removed from the needle 50 before inserting the needle 50 into a vein of the donor and blood extracted from the donor into the blood bag in the usual manner. When blood extraction is complete, the needle 50 is extracted from the donor by grasping the flattened side walls 15 of the body 14 of the barrel 10 and extracting the needle 50. While still grasping the barrel 10, the knurled surface 98 of the locking member 90 is grasped by the medical practitioner's opposing hand and rotated clockwise (as viewed from the trailing end) to an unlocking position. This disengages the hook-like members 85 of the cannula guard 70 from the locking projections 96 of the locking member 90. The compressive force applied by the spring 100 then drives the cannula guard 70 forward, deploying it to the deployed position depicted in FIG. 2, covering the needle 50. As the cannula guard 70 is driven forward, the tabs 80 of the cannula guard 70 are depressed inwardly as they engage the internal shoulders 22 of the barrel 10. As the tabs 80 pass through the opening at the leading face of the neck 13 of the barrel 10, the tabs 80 deflect outwardly, engaging the leading face of the neck 13, thereby preventing the cannula guard 70 from being pushed back into the barrel 10. The external shoulders 84 of the cannula guard 70 prevent the cannula guard 70 from being pushed beyond the deployed position and completely out of the barrel 10 as they engage the internal shoulders 22 of the barrel 10. The structures of the tabs 80 and external shoulders 84 of the cannula guard 70 thus co-operate with the structures of the leading face of the neck 13 and internal shoulders 22 of the barrel 10 to allow relative displacement of the cannula guard 70 to the deployed position, whilst preventing relative displacement of the cannula guard 70 from the deployed position back towards the retracted position. The needle 50 is thus permanently isolated from contact with the medical practitioner or any other personnel involved in processing the collected blood, thereby preventing the spread of blood-borne diseases.

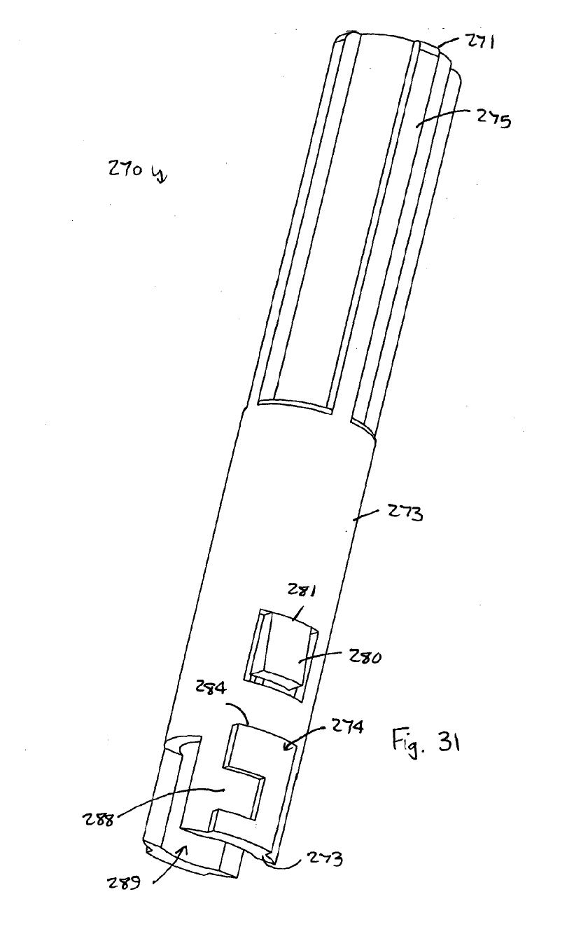

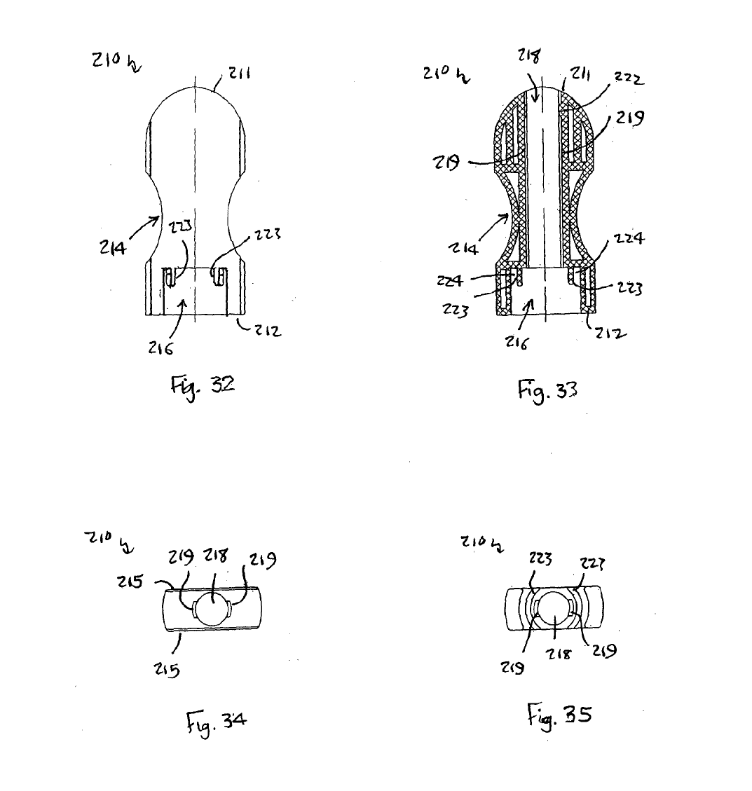

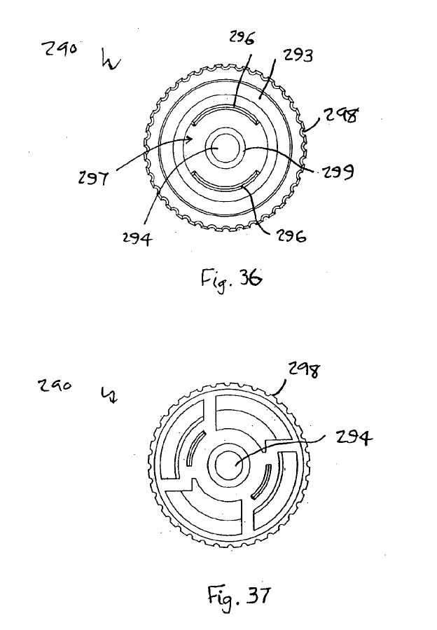

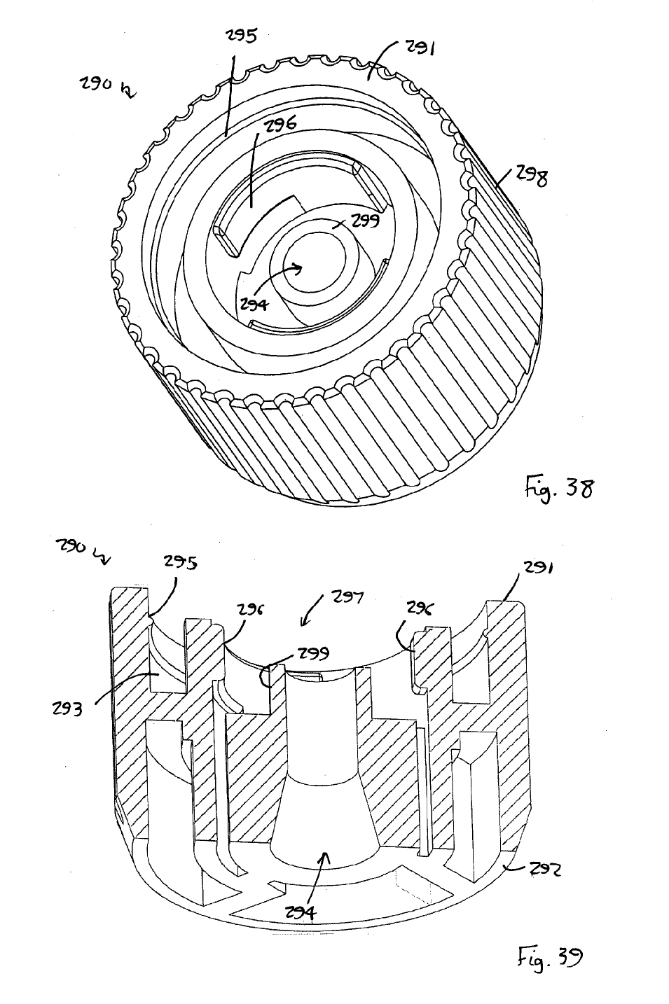

An intravenous cannula assembly 201 according to a second embodiment, again in the form of a blood bag needle assembly, is depicted in FIGS. 24 to 39. The intravenous cannula assembly 201 utilises a similar structure, mechanism and operating principles to the intravenous cannula assembly 1 of FIGS. 1 to 23. Equivalent features of the intravenous cannula assembly 201 of the second embodiment are provided with the same reference numerals as those of intravenous cannula assembly 1 of the first embodiment, increased by 200.

Referring first to FIGS. 24 through 26, the intravenous cannula assembly 201 generally comprises a barrel 210, cannula hub 230, cannula 250, cannula guard 270 and deployment mechanism including a locking member 290 and spring 300. As with the first embodiment, the cannula 250 is in the form of a blood bag needle and is mounted in, and in fluid communication with, a hub aperture 235 that extends between the hub leading end 231 and hub trailing end 232. The cannula 250 extends from the hub leading end 231 through the barrel leading end 211. The cannula guard 270 is concentrically mounted about the cannula hub 230. In the same general manner as the first embodiment, the deployment mechanism is operable to deploy the cannula guard 270 from a retracted position, depicted in FIG. 24, at which it is at least substantially located within the barrel 210, to a deployed position, depicted in FIG. 25, extending from the barrel leading end 211 and covering the portion of the cannula 250 that extends through the barrel leading end 211.

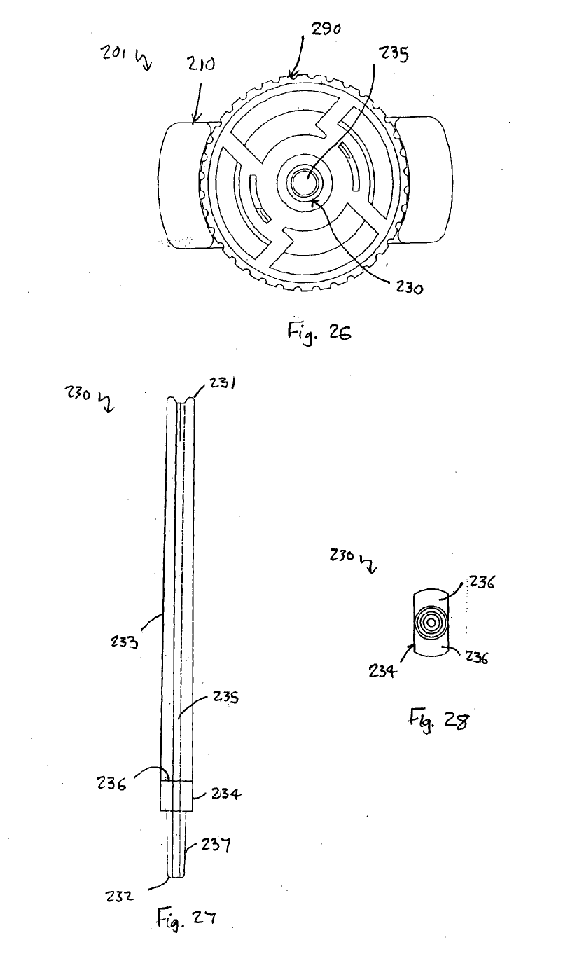

The cannula hub 230 of the second embodiment is depicted in detail in FIGS. 27 and 28. The cannula hub 230 comprises an elongate tubular hub stem 233 located towards the hub leading end 231 and a flattened hub boss 234 located towards the hub trailing end 232. A pair of opposing shoulders 236 are defined by the leading face of the flattened hub boss 234 were it meets the trailing end of the hub stem 233. A tapered tubular hub tail 237 extends between the flattened hub boss 234 and hub trailing end 232. The hub tail 237 has a tapered exterior diameter sized to receive a blood delivery tube (not depicted) such that the blood delivery tube is in fluid communication with the hub aperture 235 that extends through the length of the cannula hub 230. The hub aperture 235 is sized to receive the cannula/needle 250 at the hub leading end 231 with an interference fit so that the needle 250 remains fixed in relation to the cannula hub 230.

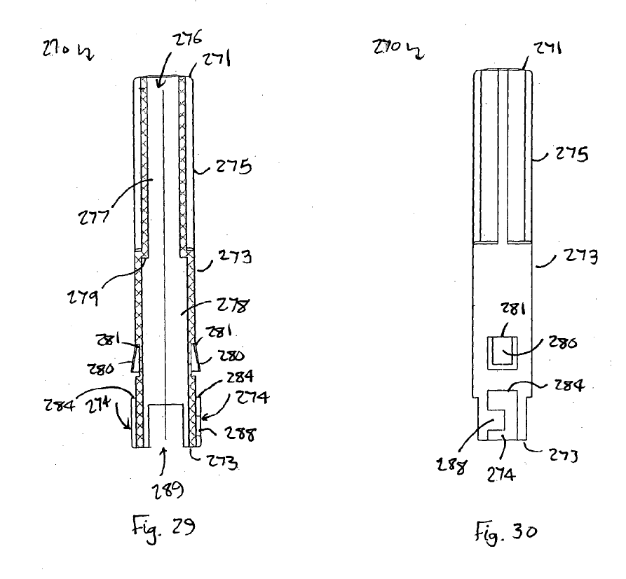

The cannula guard 270 of the second embodiment is depicted in detail in FIGS. 29 to 31. The cannula guard 270 comprises an elongate tubular stem 273 having a generally cylindrical configuration, with the leading portion adjacent the guard leading end 271 configured with a series of four elongate ribs 275 in a similar manner to those of the cannula guard 70 of the first embodiment. A guard aperture 276 extends through the length of the cannular guard 270 and has a leading portion 277 sized to received the hub stem 233 and a trailing portion 278 of greater diameter sized to receive both the hub stem 233 and the spring 300 concentrically mounted on the hub stem 233, as depicted in FIGS. 24 and 25. An annular internal shoulder 279 is defined at the intersection between the leading portion 277 and trailing portion 278 of the guard aperture 276 for restraining the leading end of the spring 300.

Two diametrically opposed tabs 280 are provided on the guard stem 273 toward the guard trailing end 272. The tabs 280 are effectively hinged from the guard stem 273 at their leading edges 281 and are biased to protrude from the stem 273 as best depicted in FIG. 29. A cut-out 289 extends through the thickness of the cannula guard 270 at the guard trailing end 272 for receipt of the flattened hub boss 234 of the cannula hub 230. Two diametrically opposed locking formations 274 are formed on the outer surface of the guard stem 273 between the tabs 280 and the guard trailing end 272. Two external shoulders 284 are defined at the intersection between the locking formations 274 and the guard stem 273. A locking recess 288 is formed in each of the locking formations 274. When viewed from the trailing end 272, each locking recess 288 extends in a clockwise direction about the longitudinal axis of the cannula guard 270.

The barrel 210 of the second embodiment is depicted in detail in FIGS. 32 to 35. As opposed to the generally cylindrical barrel 10 of the first embodiment, the barrel 210 of the second embodiment has a relatively broad, flat shape. This facilitates comfortably strapping the cannula assembly 201 to the arm of a patient with one substantially flat face 215 of the barrel 210 resting on the patient's arm. The barrel 210 has two opposing substantially flat faces 215 and a barrel aperture 218 extending through the length of the barrel 210 from the barrel leading end 211 to a mounting recess 216 that extends from the barrel trailing end 212 and through the thickness of the barrel 210 between the opposing faces 215. The barrel 210 has a central waisted portion 214 that facilitates gripping of the barrel 210. A pair of curved lugs 223 are located in the mounting recess 216 on either side of the barrel aperture 218 for mounting the locking member 290. A groove 224 extends along the radially outer surface of each of the lugs 223.

The barrel aperture 218 has a pair of slots 219 extending longitudinally along either side thereof for receipt of the tabs 280 and locking formations 274 of the cannula guard 270 such that the cannula guard 270 is longitudinally displaceable along the barrel 210 but is prevented from rotation within the barrel 210. A pair of elongate projecting detents 222 are located in the slots 219 toward the barrel leading end 211 for engaging the tabs 280 of the cannula guard 270 and the leading edge of the locking formations 274 of the cannula guard when the cannula guard is in the extended position, as depicted in FIG. 28, to form a captive locking arrangement preventing the cannula guard 270 from being pushed back into the barrel 210 or removed from the barrel 210.

The deployment mechanism includes the spring 300, which biases the cannula guard 270 towards its deployed position depicted in FIG. 25, and a releasable locking arrangement which retains the cannula guard 270 in the retracted position, as depicted in FIG. 24, prior to release of the releasable locking arrangement. The releasable locking arrangement here comprises a first interlocking structure provided on the cannula guard 270, in the form of the locking formations 274, and a locking member 290. The locking member 290 is depicted in detail in FIGS. 36 to 39. The locking member 290 has a generally cylindrical form, including a knurled peripheral surface 298 to facilitate grip. The locking member 290 has a leading end 291 and a trailing end 292. An annular outer recess 293 is formed at the leading end 291 of the locking member 290 for receipt of the lugs 223 in the recess 216 of the barrel 210, as depicted in FIGS. 24 and 25. An inwardly projecting annular rim 295 is formed in the wall of the outer recess 293 to lock the lugs 223 into the recess 293 by engaging the grooves 224 formed on each of the lugs 223. As a result, the locking member 290 is locked onto the barrel 210 whilst allowing relative rotation therebetween, with the locking member 290 being angularly displaceable about the longitudinal axis of the barrel 210.

A central recess 297 is formed at the leading end 291 of the locking member for receipt of the guard trailing end 273 and locking formations 274 of the cannula guard 270 as depicted in FIG. 24. Locking projections 296 are formed on opposing sides of the wall of the central recess 297, forming a second interlocking structure. The locking projections 296 engage the locking recesses 288 of the locking formations 274 of the cannula guard 270 to retain the cannula guard 270 in the retracted position depicted in FIG. 24.

A central locking member boss 299 extends into the central recess 297 and defines the leading portion of a central locking member aperture 294. The central locking member aperture 294 extends through the trailing end 292 of the locking member 290 for receipt of the hub tail 237 of the cannula hub 230 with the hub flattened boss 234 located on the end of the locking member boss 299.

The cannula assembly 201 will typically be provided with a standard needle cap 110 in the same manner as the cannula assembly 1 of the first embodiment.

Referring back to FIG. 24, the cannula assembly 201 is assembled with the cannula guard 270 in the retracted position. The cannula/needle 250 is mounted in the hub aperture 235 of the cannula hub 230, extending from the hub leading end 231 through the barrel leading end 211. The cannula hub 230 is in turn mounted in the barrel 210 with the hub tail 237 located in the locking member aperture 294 and the flattened hub boss 234 abutting the locking member boss 299. The locking member 290 is mounted in the mounting recess 216 of the barrel 210 with the lugs 223 of the barrel 210 located in the outer recess 293 of the locking member 290, with the annular rim 295 formed in the wall of the outer recess 293 engaging the grooves 224 formed on each of the lugs 223. A blood delivery tube (not depicted) is mounted on the hub tail 237. The cannula guard 270 is concentrically mounted about the cannula hub 230 within the barrel 210 in the retracted position with the leading end 271 of the cannula guard 270 located within the barrel aperture 218. The locking projections 296 of the locking member 290 engage the locking formations 274 of the cannula guard 270, with the locking projections 296 located within the locking recess 288, to retain the cannula guard 270 in the retracted position. The locking member 290 projects laterally beyond each opposing flat face 215 of the barrel 210 to provide ready access to the knurled peripheral surface 298 of the locking member. The spring 300 is concentrically mounted about the hub stem 233 of the cannula hub 230 within the guard aperture 276 of the cannula guard 270. The leading end 301 of the spring 300 bears against the shoulder 279 in the guard aperture 276 of the cannula guard 270 whilst the trailing end 302 of the spring 300 bears against the shoulders 236 defined by the leading face of the hub boss 234. The spring 300 imparts a driving force on the cannula guard 270 tending to deploy it towards the deployed position, however deployment is prevented by virtue of engagement of the locking formations 274 of the cannula guard 270 and the locking projections 296 of the locking member 290. The needle cap 110 is mounted over the needle 250.

The cannula assembly 201 is utilised in the same general manner as the cannula assembly 101 of the first embodiment. With the cannula assembly 201, when blood extraction is complete, the needle 250 is extracted from the donor by grasping the waisted portion 214 of the barrel 210 and extracting the needle 250. Whilst still grasping the barrel 210, the knurled surface 298 of the locking member 290 is grasped by the medical practitioner and rotated in an anti-clockwise direction (as viewed from the trailing end) to an unlocked position, disengaging the locking projections 296 from the locking recesses 288 and releasing the cannula guard 270. The compressive force applied by the spring 300 then drives the cannula guard 270 forward, deploying it to the deployed position depicted in FIG. 25, covering the cannula/needle 250.

As the cannula guard 270 is driven forward, the tabs 280 of the cannula guard 270 are depressed inwardly as they engage the detents 222 toward the barrel leading end 211. As the tabs 80 pass the detents 222, the tabs 280 deflect outwardly engaging the leading edge of the detents 222, thereby preventing the cannula guard 270 from being pushed back into the barrel. The structures of the tabs 280 and locking formations 274 of the cannula guard 270 thus co-operate with the structures of the detents 222 of the barrel 210 to allow relative displacement of the cannula guard 270 to the deployed position, whilst preventing relative displacement of the cannula guard 270 from the deployed position back towards the retracted position. The needle 270 is thus permanently isolated from contact with the medical practitioner or any other personnel involved in processing the collected blood, thereby preventing the spread of blood-borne diseases. The external shoulders 284 defined by the locking formations 274 of the cannula guard 270 prevent the cannula guard 270 being pushed beyond the deployed position and completely out of the barrel 210 as they engage the detents 222 of the barrel 210.

Whilst the cannula assembly has been described in relation to a blood bag needle assembly, the cannula assembly described may be equally utilised for other intravenous cannula operations such as intravenous drug and/or nutrient delivery by drip, blood transfusions and the like.

1. An intravenous cannula assembly comprising:
a longitudinally extending barrel having a barrel leading end and a barrel trailing end;
a cannula hub mounted in said barrel and having a hub leading end, a hub trailing end and a hub aperture extending between said hub leading end and said hub trailing end, said cannula hub being adapted to mount a tube at said hub trailing end in fluid communication with said hub aperture;
a cannula mounted in, and in fluid communication with, said hub aperture, said cannula extending from said hub leading end through said barrel leading end;
a cannula guard concentrically mounted about said cannula hub; and
a deployment mechanism adapted to deploy said cannula guard from a retracted position at least substantially within said barrel to a deployed position extending from said barrel leading end and covering the portion of said cannula extending through said barrel leading end.

2. The assembly of claim 1, wherein said cannula guard engages said barrel adjacent said barrel leading end for all positions of said cannula guard between said retracted position and said deployed position.

3. The assembly of claim 2, wherein said barrel engages said cannula guard toward a trailing end of said cannula guard for all positions of said cannula guard between said retracted position and said deployed position.

4. The assembly of claim 2, wherein a leading portion of said cannula guard is provided with a series of longitudinally extending ribs spaced about a periphery of said leading portion of said cannula guard, said ribs engaging said barrel adjacent said barrel leading end.

5. The assembly of claim 1, wherein said deployment mechanism comprises:
a spring biasing said cannula guard towards said deployed position; and
a releasable locking arrangement adapted to retain said cannula guard in said retracted position until release of said releasable locking arrangement.

6. The assembly of claim 1, wherein said releasable locking arrangement comprises:
a first interlocking structure provided on said cannula guard;
a locking member mounted on said barrel and having a second interlocking structure;
said locking member being displaceable between a locking position at which said first interlocking structure engages said second interlocking structure to thereby retain said cannula guard in said retracted position and an unlocking position at which said first interlocking structure disengages from said second interlocking structure, thereby releasing said cannula guard.

7. The assembly of claim 6, wherein said first interlocking structure is located within said barrel.

8. The assembly of claim 6, wherein said locking member is of a generally cylindrical form and is angularly displaceable about a longitudinal axis of said barrel between said locking position and said unlocking position.

9. The assembly of claim 6, wherein said locking member is mounted on said barrel adjacent said barrel trailing end.

10. The assembly of claim 9, wherein said barrel has a pair of opposing substantially flat faces, said locking member protruding laterally beyond each of said faces.

11. The assembly of claim 8, wherein said cannula hub is mounted on said locking member.

12. The assembly of claim 1, wherein said cannula hub and said cannula are each longitudinally fixed in relation to said barrel.

13. The assembly of claim 1, wherein said cannula guard is rotationally fixed in relation to said barrel.

14. The assembly of claim 1, further comprising a captive locking arrangement adapted to captively retain said cannula guard in said deployed position after deployment.

15. The assembly of claim 14, wherein said captive locking arrangement comprises co-operating structures of said cannula guard and of said barrel configured to allow relative displacement of said cannula guard to said deployed position and to prevent relative displacement of said cannula guard from said deployed position towards said retracted position.

16. The assembly of claim 15, wherein said captive locking arrangement comprises:
a pair of tabs provided on opposing sides of said cannula guard, each said tab having a leading edge hingedly connected to a stem of said cannula guard and biased to protrude from said stem; and
a co-operating structure of said barrel adapted to engage said tabs, deflecting said tabs inwardly, as said cannula guard is deployed toward said deployed position, and to engage a trailing end of each said tab when said cannula guard is in said deployed position, preventing displacement of said cannula guard from said deployed position toward said retracted position.

17. The assembly of claim 16, wherein said co-operating structure of said barrel comprises a neck of said barrel defining a reduced cross-section leading portion of said barrel aperture.

18. The assembly of claim 16, wherein said co-operating structure of said barrel comprises a pair of opposing detents projecting into said barrel aperture.

19. The assembly of claim 15, wherein said captive locking arrangement further comprises an external shoulder formed on said cannula guard toward a trailing end of said cannula guard, said shoulder being adapted to engage a co-operating structure of said barrel to prevent said cannula guard from being deployed beyond said deployed position.
